(19) United States
(12) Patent Application Publication
(10) Pub. No.: US 2007/0238941 A1
(43) Pub. Date: Oct. 11, 2007

(54) Methods and devices for evaluating skin

(76) Inventors: Janeta Nikolovski, Princeton, NJ (US); Benjamin C. Wiegand, Yardley, PA (US); Georgios N. Stamatas, Paris (FR); Nikiforos Kollias, Skillman, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/390,760

(22) Filed: Mar. 28, 2006

(51) Int. Cl. A61B 5/00

(52) U.S. Cl. 600/306

(57) ABSTRACT

Provided are methods and devices for evaluating skin by introducing a fluid comprising water to the skin; measuring at least one skin barrier property that correlates to the water content of the skin over time, the measuring step comprising taking at least one measurement of the skin barrier property in the time frame starting from completion of the introducing step up to about 60 seconds after such introducing step and optionally taking a plurality of skin barrier property measurements after about 60 seconds after completion of the introducing step; and comparing the skin water content measurements of the measuring step with at least one Comparative Data Set to determine the relative infant-like barrier properties of the skin.

### FIELD OF INVENTION

The present invention relates to methods and devices for evaluating the skin. More specifically, the present invention relates to methods and devices for measuring barrier properties of skin over time and comparing such measurements with key measurements of comparative infant and/or adult skin samples to determine the relative infant-like properties of the measured skin.

### BACKGROUND

A variety of conventional methods for measuring properties of the skin, such as transepidermal water loss, wrinkles, lines, pigmentation, water content, elasticity, and the like are known. Many of such conventional methods have been used to develop products and methods of changing skin properties (or the consumer perception of skin properties) to be more desirable/acceptable to the consumer. For example, certain skin properties often considered undesirable, such as wrinkles, pigmentation, and the like, in adult skin have been measured and compared to “youthful” skin (often skin of people ages 12 to 25, for example, wherein lower levels of the negative properties tend to be found), and such comparisons have then been used to develop and support claims of consumer solutions for making adult skin appear more like “youthful” skin. Other studies have measured and compared adult skin to newborn skin (the skin of babies up to about 1 month old), for example, as described in “Dry Skin of Newborn Infants: Functional Analysis of the Stratum Corneum,” Shinobu Saijo, M.D., and Hachiro Tagami, M.D. Pediatric Dermatology, Vol. 8, No. 2, pps. 155-159 (June 1991).

Applicants have recognized, however, that there is a need in the art for methods of effectively evaluating properties of skin, for example, skin barrier properties, as compared to properties of infant skin, to allow for the assessment of how “infant-like” is a particular sample of skin and for the development of products and methods of making the skin more or less “infant-like” and/or “baby-like.” In particular, applicants have noted that conventional methods of skin assessment tend to lack any recognition that the skin properties of infants, as compared to newborns and/or youthful skin, tend to be unique, and such methods fail to provide any comparative differences between infant and non-infant skin suitable for use in comparing whether skin is more or less “infant-like.”

### SUMMARY

The present invention provides methods and devices for evaluating the skin that overcome the disadvantages of the prior art. In particular, applicants have discovered unexpected and significant differences in the skin barrier properties of infant skin as compared to adult and/or newborn skin which allow for effective evaluation of the relative infant-like barrier properties of skin. In addition, applicants have recognized that significant differences in the barrier properties of infant skin as compared to non-infant skin, for example properties that correlate to the ability of the skin to retain water over time, occur within a particular time frame after the introduction of water thereto, and thus, measurement and comparison of at least one or more measurements occurring within this time frame can provide significant improved ability to evaluate a skin sample for its relative level of “infant-like” properties, such as skin barrier properties.

Therefore, according to one aspect, the present invention relates to methods of evaluating skin comprising the steps of introducing a fluid comprising water to the skin; measuring at least one skin barrier property that correlates to the water content of the skin over time, the measuring step comprising taking at least one measurement of the skin barrier property in the time frame starting from completion of the introducing step up to about 60 seconds after such introducing step and optionally taking a plurality of skin barrier property measurements after about 60 seconds after completion of the introducing step; and comparing the skin water content measurements of the measuring step with at least one Comparative Data Set to determine the relative infant-like barrier properties of the skin.

According to another aspect, the present invention comprises systems and devices for evaluating the skin comprising an energy source, a detector operatively associated with the energy source, a comparing component for determining the relative infant-like barrier properties indicated by the measured data; and a visual or audio display for displaying an indication of relative infant-like barrier properties associated with the measured data.

### BRIEF DESCRIPTION OF THE DRAWING

FIG. 1 is a graphical depiction of skin barrier properties associated with both infant and adult skin measured in accord with one embodiment of the present invention.

### DESCRIPTION OF PREFERRED EMBODIMENTS

As used herein, the term “infant skin” refers in general to the skin of a human infant or baby, preferably of about the age of 1 month to 5 years. In certain preferred embodiments, infant skin is skin of a human infant or baby of about the age of 1 month to about 4 years, preferably in age of from about 3 months to about 3 years old, more preferably from about 3 months to about 2 years old, more preferably from about 3 months to about 1 year old. The term “newborn skin” refers in general to the skin of a human baby in age of from birth to 1 month, preferably less than 1 month.

The term “adult skin” refers in general to the skin of a human that is older than about 5 years old, preferably about 15 years old or older, more preferably about 25 years old or older. In certain other preferred embodiments, adult skin refers to skin of a human of about 40 years or older, including from about 40 to about 65 years old, or from about 65 and older.

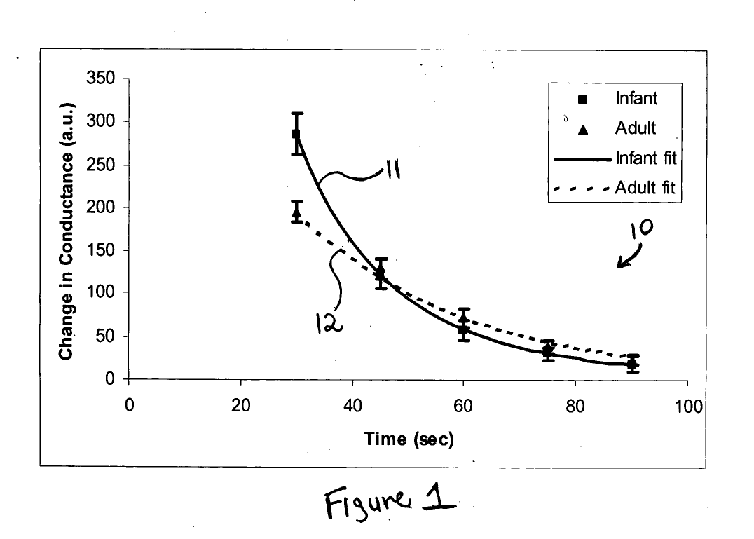

As noted above, applicants have discovered, to applicant's knowledge, heretofore unknown differences in the skin barrier properties of infant skin as compared to adult skin and/or newborn skin, including differences in the ability of such skin to retain water therein over time, and have developed the present novel methods and devices for evaluating skin to determine the relative infant-like barrier properties associated therewith. In particular, applicants have found that infant skin tends to both absorb and desorb water at significantly faster rates than adult skin (which adult skin, in turn, has been shown in the art to absorb and desorb water at faster rates than newborn skin). For example, shown in FIG. 1 is a graphical representation 10 of the data obtained by measuring the conductance/water content of infant skin over time (illustrated by curve 11) and adult skin (illustrated by curve 12) in accord with one embodiment of the present invention. Comparison of curves 11 and 12 show distinct and measurable differences in the barrier properties between the adult and infant skin data, especially in the time frame of about 60 seconds or less from the introduction of fluid (at time zero), and within about 60 seconds or less from a blotting step (not shown) which occurred at time 10 seconds. Specifically, as shown in the graph, infant skin tends to absorb and desorb water significantly faster (at higher rates) than adult skin (unlike newborn skin which tends to adsorb and desorb water slower than adult skin). After the aforementioned time period, at least in the embodiment shown in FIG. 1, the differences in barrier properties tend to be less significant. Accordingly, applicants have recognized that methods of evaluating the skin and skin barrier properties which incorporate the comparison of measured data including data within the aforementioned key time frame with comparable data including data within the aforementioned key time frame allow for more effective evaluation of skin barrier properties as compared to conventional skin evaluation methods. Moreover, applicants' unexpected discovery of the distinct properties associated with infant skin allow for the evaluation of the relative infant-like barrier properties associated with measured skin, wherein, as will be recognized in light of the teachings herein, a skin sample exhibiting higher rates of absorbtion/desorbtion (preferably both) of water tends to have a relatively more infant-like barrier than skin exhibiting slower rates of absortion and/or desorbtion. Thus, applicants have developed the present effective new methods for evaluating the skin comprising the steps of introducing a fluid comprising water to the skin; measuring at least one skin barrier property that correlates to the water content of the skin over time, the measuring step comprising taking at least one measurement of the skin barrier property in the time frame starting from completion of the introducing step up to about 60 seconds after such introducing step and optionally taking a plurality of skin barrier property measurements after about 60 seconds after completion of the introducing step; and comparing the skin water content measurements of the measuring step with at least one Comparative Data Set to determine the relative infant-like barrier properties of the skin.

The introducing step of the present methods may comprise introducing any of a variety of fluids comprising water in any of a variety of suitable manners to the skin in accord with the present invention. Any fluid from which water may be absorbed into the skin upon introduction of the fluid thereto may be used in the present methods. Examples of suitable fluids include water (distilled, tap, and the like), combinations of water with other fluids and/or solids, such as, glycerin, physiologic and/or buffered fluids such as saline, combinations of two or more thereof, and the like. In certain preferred embodiments, the fluid is water.

Any suitable manner for introducing the fluid to a skin sample may be used in accord with the introducing step. For example, the fluid may be contacted, sprayed, pumped, poured, droppered, splashed, or wiped onto the skin, the skin may be immersed into the fluid, combinations of two of more such methods, and the like. In light of the disclosure herein, those of skill in the art will be readily able to adapt various methods for introducing a fluid to the skin for use in the present invention.

Any suitable amounts of fluid may be introduced to the skin in accord with the introducing step. In certain preferred embodiments, sufficient amount of fluid is introduced to the skin such that after about 10 seconds from introducing the fluid, at least a portion of said fluid remains visible on the surface of the skin. In certain other preferred embodiments, fluid is introduced to the skin in an amount such that after about 10s from introducing the fluid, essentially no fluid is visible on the surface of the skin, that is, substantially all of the fluid is either absorbed in, and/or evaporated from, the skin.

In those embodiments of the present invention wherein some of the introduced fluid remains visible on the skin after about 10s from introducing the fluid, the introducing step further comprises the step of drying the remaining visible fluid from the skin. Any suitable means for drying the fluid may be used including, for example, blotting, blowing, evaporating, shaking, combinations of two or more thereof, and the like. In certain preferred embodiments, the introducing step comprises blotting visible fluid remaining after 10s from the skin. In those embodiments of the present invention wherein water is removed from the skin after contacting the skin with such water, completion of the introducing step occurs after such removal step.

As will be recognized by those of skill in the art, a variety of methods for measuring the water content of the skin exist, including methods of directly and/or indirectly measuring the water content of skin, such as, for example, via Raman (wherein incident monochromatic laser light induces inelastic light scattering resulting in highly specific information on the molecular concentration profiles of the skin, i.e. water content), by measuring the conductance of electricity through the skin, the capacitance and/or impedance of the skin, and the like, which measures are readily correlated to the amount of water in the skin. Accordingly, as used herein, a “skin barrier property that correlates to the water content of the skin,” measured in accord with the present invention, refers generally to the water content of the skin and any other property that can be readily correlated and/or converted to the water content of the skin. Certain preferred skin barrier properties that correlate to the water content of the skin include, the water content of the upper layers of the skin including the stratum corneum, as well as, conductance, capacitance, impedance, Fourier Transform Infrared (FTIR), Near Infrared (NIR), ATR-FTIR, of the upper layers of the skin including the stratum corneum.

Any suitable method for measuring the skin barrier properties that correlate to the water content of the skin, and change therein over time, may be used in accord with the present invention. Examples of suitable methods include direct methods of measuring water content and methods of measuring one or more skin properties that correlate to water content in the skin, include Raman Confocal, NIR, FTIR, ATR-FTIR, methods of measuring conductance, capacitance, impedance, combinations of two or more thereof, and the like. By way of reference, certain examples of such measurements are described in the following articles which are incorporated herein by reference: Zhang, S. L., et al. “Near infrared imaging for measuring and visualizing skin hydration. A comparison with visual assessment and electrical methods”, Journal of biomedical Optics, Vol. 10, Issue 3, 031107 (May/June 2005); Caspers P J, et al. “In vivo confocal Raman microspectroscopy of the skin: noninvasive determination of molecular concentration profiles” Journal of Investigative Dermatology 116, 434-442 (2001); and Rim, J. H. et al. “Electrical measurement of moisturizing effect on skin hydration and barrier function in psoriasis patients” Clin Exp Dermatol. 30(4):409-13 (July 2005). Certain preferred measurement methods include measures of conductance, capacitance, impedance, Raman Confocal, FTIR, combinations of two or more thereof, and the like. Preferably, the measuring step comprises measuring the water content of the upper layers of skin including the stratum corneum. In certain other preferred embodiments, the measuring step comprises measuring the water content of the upper layers of skin including the stratum corneum and layers of the upper epidermis. In light of the description herein, those of skill in the art will recognize and readily be able to adapt any of a variety of conventional means of measuring skin barrier properties that correlate to water content of the skin for use in the measuring steps of the present invention.

Any number of suitable measurements of skin barrier properties may be taken over time in accord with the present invention. In preferred embodiments, the measuring step comprising taking at least one skin barrier measurement in the time frame up to 60 seconds from the introduction of fluid to the skin via the introducing step. In certain more preferred embodiments, at least one measurement is taken in the time frame up to 45 seconds, more preferably up to 40 seconds, more preferably up to 30 seconds after the introduction of fluid to the skin via the introducing step. In certain more preferred embodiments, at least a plurality of measurements are taken in the time frame up to 60 seconds from the introduction of fluid to the skin via the introducing step. Preferably, at least one of such plurality of measurements of the measuring step comprises a measurement taken in the time frame of up to 30 seconds after the introduction of water to the skin via-the- introducing step. In certain other preferred embodiments, the aforementioned plurality of measurements taken up to about 60, 45, or 40 seconds after introduction of fluid comprises at least three or more measurements, preferably at least four or more measurements.

In certain embodiments, the measuring step further includes the step of taking one or more measurements of skin barrier properties that correlate to water content of the skin more than 60 seconds after the introduction of fluid to the skin via the introduction step. In certain preferred embodiments, the measuring step includes taking at least two, and preferably three or more measurements of the water content of the skin over time in the period more than 60 seconds after the introducing step.

In light of applicants discovery that infant skin and adult skin exhibit significant measurable differences in skin barrier properties in the time frame from the introduction of fluid to the skin up to about 60 seconds thereafter, the present methods further comprise the step of comparing the skin barrier measurements of a skin sample taken via the measuring step of the invention to a set of skin barrier data correlating to comparable measurements of an infant skin and/or adult skin standard, including data taken from the time frame of up to 60 seconds after introduction of a fluid to the comparable skin standard, to determine the relative infant-like barrier properties of the measured skin data to the comparable data. As used herein, the term “Comparative Data Set” refers to a set of skin barrier property data for one or more infant skin and/or adult skin standard(s), against which the subject skin sample, measured in accord with the present invention, is to be compared, which set of data includes data for such standard measured from within the time frame of up to 60 seconds of introduction of a fluid comprising water to such standard. That is, for example, in embodiments of the present invention comprising measuring the water content of a subject skin sample over time, a Comparative Data Set for use in evaluating such subject skin sample preferably comprises measurements of the water content over time for at least one infant skin standard, at least one adult skin standard, or combinations of two or more thereof, wherein the measurements for the standard(s) include measurements from, or data correlating to measurements from, within a time frame of up to 60 seconds from the introduction of a fluid to the standard(s).

The skin barrier measurements taken in accord with the measuring step of the present invention and the data of the Comparative Data Set may be in any suitable format for comparison in accord with the comparing step of the present invention. For example, the measured data and comparative data may each individually comprise data in visual formats, including but not limited to, numerical (or other symbolic) data arranged in one or more lists, tables, charts, matricies, equations, other compilations, and the like, graphical representations of data including graphs (fitted line, bar, pie, combinations of two or more thereof, and the like), maps, images, spectra, as well as, auditory formats (for example, data converted to tones wherein different data creates different tones), or any other format in which difference can be perceived to detect similarities/differences between the measured data and the standard data, combinations of two or more thereof, and the like. In preferred embodiments, the measured data and data of the Comparative Data Set are compared in visual formats including lists, tables, charts, matricies, equations, other compilations of numerical data, and graphical representations of data including graphs (fitted line, bar, pie, combinations of two or more thereof, and the like), maps, images, combinations of two or more thereof, and the like.

Any method for comparing measurements taken in accord with the measuring step and data of a Comparative Data Set to determine relative infant-like properties of the measured data may be used in accord with the present invention. For example, two or more sets of data including at least one set of measured data and at least one set of comparative data may be compared visually, such as by visual comparison of lists, tables, etc. of numbers, comparison graphical representations of the data, comparison of data lists, tables, etc. versus graphical representation(s) of another set of data, comparison of two auditory formats, comparison of visual and auditory formats, and any other methods for comparing formats of the data from which a user can detect the differences in the data sets.

In certain preferred embodiments, the comparing step of the present invention comprises comparing a set of measurements taken in accord with the measuring step with a Comparative Data Set comprising skin barrier data for infant skin. In certain other preferred embodiments, the comparing step of the present invention comprises comparing a set of measurements taken in accord with the measuring step with a Comparative Data Set comprising skin barrier data for adult skin. In certain more preferred embodiments, the comparing step of the present invention comprises comparing a set of measurements taken in accord with the measuring step with a Comparative Data Set comprising skin barrier data for both infant skin and adult skin.

For the purposes of illustration, without intending to be limiting, applicants note that in certain preferred embodiments the Comparative Data Set comprises a set of measurements of a skin barrier property that correlates to water content taken over time from infant skin (to provide an infant standard), and the measurements taken in accord with the present invention are for adult skin. Accordingly, the comparing step may then comprise comparing the measured set of data with the set of standard infant skin data or producing a fitted line graph with a curve for the measured data and a curve for the Comparative Data Set to determine how close or disparate is the set of measured adult data (in particular the rates of water absorbtion/desorbtion) from the comparative infant set, i.e. how relatively “infant-like” or not is the measured skin sample. In other preferred embodiments, the Comparative Data Set comprises a set of measurements of a skin barrier property that correlates to water content taken over time from infant skin and adult skin (to provide an infant standard and adult standard). Accordingly, the comparing step may then comprise comparing the measured set of data with the set of standard infant skin and adult skin data or producing a fitted line graph with a curve for the measured data, a curve for the infant skin data of the Comparative Data Set, and a curve for the adult skin data of the Comparative Data Set to determine how the set of measured data compares to both the comparative infant set and adult set, i.e. how relatively “infant-like” or “adult-like” is the measured skin sample.

In certain other preferred embodiments, the comparing step comprises comparing slopes and/or rate constants associated with graphical representations of the measured data and the Comparative Data Set. For example, as shown in FIG. 1, applicants have recognized that in certain embodiments, the water content of adult skin tends to change in accord with a single rate constant from about the time that water is introduced to the skin out to longer periods of time, while water content in infant skin changes according to two different rate constants (and initial rapid change followed by a slower change similar in rate to adult skin). Accordingly, in certain embodiments, the comparing step may comprise calculating and comparing slopes of all or parts of the measured data and Comparative Data Set, or may involve fitting the data with one or more exponential curves, calculating the constants associated therewith, and comparing such constants to determine whether measured data is more or less infant-like. In particularly preferred embodiments, the comparing method comprises fitting the measured data to Equation 1 in accord with the Equation Constant Fit Method, as described and shown in Example 1, determining A and/or B and comparing such constants to a Comparative Data Set of standard A and/or B values (an example of such a Comparative Data Set is shown in Example 1).

In light of the description of preferred embodiments of comparing steps herein, those of skill in the art will be readily able to adapt a variety methods for comparing measured data with comparative data to determine the relative infant-like barrier properties associated therewith in accord with the present invention.

The present invention further provides methods of diagnosing and/or treating the skin comprising evaluating the skin in accord with the methods described hereinabove, and diagnosing a skin deficiency/abnormality and/or treatment regimen for the skin based on the evaluation. Any of a variety of skin deficiencies/abnormalities and/or treatment regimens may be assessed and provided in accord with the present methods. For example, dry skin, psoriasis, eczema, wound or ablation of the skin surface layers, diaper or other dermatitis, dermabrasion, laser treatments, sun or other burn, photodynamic therapy, combinations of two or more thereof and the like. In certain preferred embodiments, the present methods comprise evaluating the skin and identifying compositions and/or treatment(s) for making the skin barrier more infant-like, or more adult-like. In certain preferred embodiments, the treatment regimen comprises applying a composition topically to the skin and/or ingesting an ingestible composition for treating the skin.

The present invention further provides methods of evaluating the effectiveness of a particular composition or treatment of the skin in changing the skin barrier properties of a skin sample. In certain embodiments, such methods comprise evaluating the skin in accord with the methods described hereinabove, applying a composition to and/or otherwise treating the skin, and subsequently re-evaluating the skin via the aforementioned methods of the invention to determine if and how the barrier properties of the subject skin have changed. Such methods may be used to assess the effectiveness of any of a variety of compositions, such as personal care compositions, including moisturizers, cleansers, anti-acne, anti-aging, wound healing compositions and devices, and the like. The effectiveness of other methods of treating or abrading the skin, including wound dressings and bandages, tape stripping, patches, dermabrasion, microneedles, and the like may be suitable assessed via the methods of the present invention.

The present invention further provides devices for evaluating the skin comprising an energy source, a detector operatively associated with the energy source, a comparing component associated with the detector, and a display, the device being capable of measuring at least one skin barrier property that correlates to the water content of the skin over time of a skin sample to which a fluid comprising water has been applied and displaying a relative infant-like skin barrier indication. For the purposes of the present invention, an energy source and detector are considered to be “operatively associated” when connected or arranged in a manner such that energy produced by the energy source is capable of being introduced to the skin and subsequently detected as energy, in the same or a different form, by the detector. For example, without intending to be limiting, a light source and detector of the present invention may be operatively associated in any manner such that excitation energy from the light source is transmitted to and absorbed by the skin, and the energy produced by the excited skin is transmitted to, and detected by, the detector. For the purpose of further non-limiting illustration, other light, electric or sound energy from an energy source may be introduced to the skin through which it travels, or from which it is reflected, to the detector where it is detected.

Any of a wide range of energy sources suitable for introducing energy (excitation, fluorescent, electric, sound, heat, combinations of two or more thereof, and the like) to human skin may be used in the devices of the present invention. Examples of suitable energy sources include light sources, sound sources, electricity sources, mechanical, chemical, combinations of two or more thereof, and the like. As used herein, the term “light source” refers to a source of optical radiation, whether ultraviolet, visible or infrared. Suitable non-limiting examples of light sources include an argon laser, blue laser, tunable laser, light emitting diodes (LED), incandescent lamp, combinations of two or more thereof, and the like. Non-limiting examples of sound sources include ultrasound device, speaker system, combinations of two or more thereof, and the like, and non-limiting examples of electricity include DC or AC sources, combinations of two or more thereof, and the like. Suitable examples of other energy sources include vibration, chemical reaction on or in the skin as a source of energy, combinations of two or more thereof, and the like.

Any of a wide range of suitable detectors can be used according to the present invention. Non-limiting examples of suitable detectors include focal plane array, spectrophotometer, spectrometer (gas or mass), photomultiplier tube, CCD camera or array equipped with a monochromator, filters, the naked eye, combinations of two or more thereof, and the like.

Any of a variety of comparing components capable of comparing the data detected and comparing it to comparable data to determine relative infant-like barrier properties associated with the data may be associated with a detector and used in the present devices. The comparing component and detector may be associated via any conventional means (cable connection, optical connection, wireless, and the like) such that data detected by the detector may be transmitted/transferred to the comparing component. Examples of suitable comparing components include computers, one or more processors or microprocessors, combinations of two or more thereof, and the like. In certain preferred embodiments, the comparing component comprises a memory for storing comparative data (i.e. one or more Comparative Data Sets) for use in comparing and evaluating detected data relative to the stored comparative data. In certain other preferred embodiments, the comparing component is programmable to accept or change the comparative data stored therein. In certain embodiments, the comparing component may store data detected in one use of the present device for comparison to a subsequent set of data detected in a subsequent use of the same device.

Any of a variety of conventional displays may be associated with the present devices to display a message, symbol, etc. to a user. The display may be associated via any conventional means (cable connection, optical connection, wireless, and the like) such that data may be transmitted to the display to indicate what message, symbol, color, printout, etc. is to be displayed. Preferably, the display is capable of communicating to a user a relative measure of infant-like barrier properties based on detections made via the device detector.

In certain preferred embodiments, the device further comprises a reservoir for storing a liquid comprising water. Preferably, the reservoir comprises an opening through which the water may be introduced to the skin.

In certain preferred embodiments, the device comprises a drying component for removing excess water from the skin prior to measuring water content of the skin barrier. Examples of suitable drying components include an absorbent blotter, a fan, combinations of two or more thereof, and the like.

The device of the present invention may be configured such that after application of water to the skin, several discrete water content measurements may be detected and stored by the device for subsequent comparison in the comparing component. In certain embodiments, the device may be held against the skin for a period of time and programmed to take periodic measurements while held against the skin for use in the comparing component. In yet other embodiments, the device may be configured such that upon contact to the skin by a user (with or without activation, i.e. via switch, button, or the like), the device measures a baseline water content, introduces water to the skin, dries the excess water, takes periodic measurements, compares such measurements, evaluates the relative infant-like barrier properties indicated by the measurements, and displays a result, without any further activity on the part of the user.

In light of the disclosure herein, those of skill in the art will be readily able to configure any of a variety of devices in accord with the present invention without undue experimentation.

The invention illustratively disclosed herein suitably may be practiced in the absence of any component, ingredient, or step which is not specifically disclosed herein. Several examples are set forth below to further illustrate the nature of the invention and the manner of carrying it out. However, the invention should not be considered as being limited to the details thereof.

### EXAMPLES

The following Examples are intended to be illustrative and not limiting in any manner.

### Example 1

Examples of Comparative Data Sets comprising key conductance data for both infant skin and adult skin in accord with certain embodiments of the present invention was generated as follows:

The change in conductance over time for 88 infants (aged 3-46 months) and 97 adults (aged 14-74 years) were measured for each subject in accord with the following procedure. A baseline conductance reading of the skin on the lower dorsal arm was measured using a NOVA DPM meter (available commercially from NOVA, Portsmouth, N.H.). Thereafter, 1 drop of water (about 250 microliters) was added to a lcm area of skin on the lower dorsal arm to saturate the upper layers of skin in such area (i.e. such that after 10 seconds, free water remained visible on the skin). Ten seconds after application of such water to the skin, the wetted area was blotted dry with a paper towel. Serial conductance measurements of the blotted area were subsequently taken at 15 second intervals starting at 30 seconds after initial application of water up to 180 seconds after initial application of water.

The resulting raw data was listed in a table to form one Comparative Data Set. In addition, the measured conductance values at 30s, 45s, 60s, 75s, and 90s for the infant subjects were averaged and the average values were plotted on a line graph as shown in FIG. 1 as curve 11. The measured conductance values at 30s, 45s, 60s, 75s, and 90s for the infant subjects were averaged and the average values were plotted on the same line graph as shown in FIG. 1 (curve 12) to form a graphical Comparative Data Set.

Applicants discovered that the water desorption data for infants showed an initial rapid decay (with time constant τ₁=12 sec) and a secondary slower phase (τ₂=30 sec). The sum of two exponentials, including significant contribution from the rapid decay, was required to fit the infant data. The data for adults could be fitted well with a single exponential (with time constant τ=30 sec), with seemingly relatively little contribution from an initial rapid decay time constant. This implies that the rate of water desorption from infant skin follows a different mode than in adults, including an initial rapid process and a secondary slower one.

Equation Constant Fit Method: To effectively use the above learning for evaluating relative infant-like barrier properties of skin, applicants calculated a Comparative Data Set of rate constants by fitting the measured infant data, and separately fitting the measured adult data, to the following Equation (Equation I) and calculating the average value of A for infants and the average value for adults:
Conductance at time t=Conductance at t0=30 sec×[A×exp(−k1/T)+B×exp(−k2/T)] (I)

where T=t−t0=t−30; k1 is 12; k2 is 30;

with the condition that A+B=1 and 0<A<1, 0<B<1

Applicants calculated the value of constant A for infants to be about 0.5 or greater, on average about 0.6, and calculated the value of constant A for adults to be less than about 0.5, on average about 0.4. Accordingly, applicants incorporated such values into a Comparative Data Set to which values of constant A calculated from skin barrier measurements fitted as above may be compared to determine the relative infant-like barrier properties associated therewith.

### Example 2

The following example illustrates the measurement and evaluation of skin in accord with certain embodiments of the present invention.

A baseline measurement of conductance on the lower dorsal arm of a subject (subject 1) is measured using a NOVA DPM meter (available commercially from NOVA, Portsmouth, N.H.). Thereafter, 1 drop of water is added to the lower dorsal arm for 10 seconds and is then blotted dry. Serial conductance measurements of the blotted area are subsequently taken at 15 second intervals starting at 30 seconds after initial application of water up to 180 seconds after initial application of water.

The resulting measurements are tabulated and compared to the tabular Comparative Data Set from Example 1. The data indicate a relative rate of desorbtion that is faster than the average comparative adult data, but slower than the average infant data. Such determination indicates subject 1 has skin barrier properties that are relatively more infant-like than the average adult (but less infant-like than the average infant).

The conductance measurements are plotted with a single curve and compared to the graphical Comparative Data Set from Example 1 (shown in FIG. 1). The plotted curve indicates a relative rate of desorbtion that is faster than the average comparative adult data, but slower than the average infant data. Such determination indicates subject 1 has skin barrier properties that are relatively more infant-like than the average adult (but less infant-like than the average infant).

The conductance measurements are fit to Equation I in accord with the Equation Constant Fit Method and the value for constant A is calculated to be about 0.55. When compared to the Comparative Data Set for constant A in Example 1, it is determined that the measured data indicates a barrier that is relatively more infant-like than the average adult skin.

### Example 3

The following example illustrates the measurement and evaluation of skin in accord with certain embodiments of the present invention.

After the evaluation in Example 2, a personal care product is applied to the measured arm of subject 1. Thereafter, a baseline measurement of conductance and subsequent serial conductance measurements are taken as described in Example 2. The resulting measurements are tabulated, graphed, fitted via the Equation Constant Fit Method, and the data, graph, and calculated constant A and compared to a Comparative Data Set comprising the data and graphs of subject 1 measured and plotted in Example 2. Comparison of the data and graphs measured after application of the personal care product with the Comparative Data Set indicates that the post-application skin barrier properties of subject 1 tend to be more infant-like that the properties measured in Example 2.

### Example 4

The following example illustrates the measurement and evaluation of skin in accord with certain embodiments of the present invention.

A baseline measurement of conductance on the lower dorsal arm of a different subject (subject 2) is measured using a NOVA DPM meter (available commercially from NOVA, Portsmouth, N.H.). Thereafter, one drop of water is added to the lower dorsal arm for 10 seconds and is then blotted dry. Serial conductance measurements of the blotted area are subsequently taken at 15 second intervals starting at 30 seconds after initial application of water up to 180 seconds after initial application of water.

The resulting measurements are tabulated, graphed, fitted via the Equation Constant Fit Method, and the data, graph, and calculated constant A compared to a Comparative Data Set comprising all the data, graphs, and constants of subject 1 measured, plotted, and calculated in Examples 2 and 3, and the infant and adult data from Example 1. Comparison of the data, graphs, and/or constants of subject 2, each alone and/or together, indicates that subject 2 had skin barrier properties that were more infant-like than the average adult from Example 1, but less infant-like (more adult-like) than subject 1 (pre or post application of product) and the average infant from Example 1.

### Example 5

The following example illustrates the measurement and evaluation of skin in accord with certain embodiments of the present invention.

A baseline measurement of conductance on the lower dorsal arm of an infant subject (subject 3) is measured using a NOVA DPM meter (available commercially from NOVA, Portsmouth, N.H.). Thereafter, one drop of water is added to the lower dorsal arm for 10 seconds and is then blotted dry. Serial conductance measurements of the blotted area are subsequently taken at 15 second intervals starting at 30 seconds after initial application of water up to 180 seconds after initial application of water.

The resulting measurements are tabulated, graphed, fitted via the Equation Constant Fit Method, and the data, graph, and calculated constant A are each compiled to form Comparative Data Sets.

Six months later, a baseline conductance measurement and serial conductance measurements of subject 3 are measured in the same manner as described above in this Example. The resulting measurements are tabulated, graphed, fitted via the Equation Constant Fit Method, and the data, graph, and calculated constant A are compared with the aforementioned Comparative Data Sets compiled six months prior. Comparison of the newer measured data with the Comparative Data Sets indicates that the skin of infant subject 3 is becoming less infant-like (more adult-like) over time.

### Example 6

The following example illustrates the measurement and evaluation of skin in accord with certain embodiments of the present invention.

A baseline measurement of conductance on the lower dorsal arm of an infant subject (subject 4) having a wound on the lower dorsal arm is measured using a NOVA DPM meter (available commercially from NOVA, Portsmouth, N.H.). Thereafter, one drop of water is added to the wounded lower dorsal arm for 10 seconds and is then blotted dry. Serial conductance measurements of the wounded, blotted area are subsequently taken at 15 second intervals starting at 30 seconds after initial application of water up to 180 seconds after initial application of water.

The resulting measurements are tabulated, graphed, fitted via the Equation Constant Fit Method, and the data, graph, and calculated constant A are compared with the infant skin Comparative Data Sets of Example 1. Based on such comparison, the damage to the skin barrier from the wound is diagnosed and a wound treatment regimen is implemented.

The wounded arm is treated for two days in accord with the aforementioned treatment regimen and thereafter, a baseline conductance measurement and serial conductance measurements are measured in the same manner as described above in this Example. The resulting measurements are tabulated, graphed, fitted via the Equation Constant Fit Method, and the data, graph, and calculated constant A are compared with Comparative Data Sets comprising the skin barrier data compiled two days prior. Comparison of the newer measured data with the Comparative Data Sets indicates that the skin barrier of infant subject 3 is healing and returning to a relatively more infant-like state as the wound heals.

We claim:

1. A method of evaluating skin comprising the steps of introducing a fluid comprising water to the skin; measuring at least one skin barrier property that correlates to the water content of the skin over time, the measuring step comprising taking at least one measurement of the skin barrier property in the time frame starting from completion of the introducing step up to about 60 seconds after such completion of the introducing step and optionally taking a plurality of skin barrier property measurements after about 60 seconds after completion of the introducing step; and comparing the skin water content measurements of the measuring step with at least one Comparative Data Set to determine the relative infant-like barrier properties of the skin.

2. The method of claim 1 wherein the introducing step comprises introducing a fluid comprising water to the skin and subsequently drying excess fluid from the skin.

3. The method of claim 1 wherein said measuring step comprises measuring one or more property of the skin selected from the group consisting of conductance, capacitance, impedance, FTIR, NIR, Raman Confocal, and combinations of two or more thereof.

4. The method of claim 1 wherein said measuring step comprises taking at least one measurement of the skin barrier property in the time frame starting from completion of the introducing step up to about 45 seconds thereafter.

5. The method of claim 1 wherein said measuring step comprises taking at least one measurement of the skin barrier property in the time frame starting from completion of the introducing step up to about 30 seconds thereafter.

6. The method of claim 1 wherein said measuring step comprises taking a plurality of measurements of the skin barrier property in the time frame starting from completion of the introducing step up to about 60 seconds thereafter.

7. The method of claim 1 wherein said measuring step comprises taking a plurality of measurements of the skin barrier property after the time frame starting from completion of the introducing step up to about 60 seconds thereafter.

8. The method of claim 1 wherein said comparing step comprises comparing at least a portion of the measurements measured in the measuring step with a Comparative Data Set comprising infant skin barrier data.

9. The method of claim 1 wherein said comparing step comprises comparing at least a portion of the measurements measured in the measuring step with a Comparative Data Set comprising adult skin barrier data.

10. The method of claim 1 wherein said comparing step comprises comparing at least a portion of the measurements measured in the measuring step with a Comparative Data Set comprising both infant skin and adult skin barrier data.

11. The method of claim 1 wherein said comparing step comprises comparing said measurements and said Comparative Data Set in visual format.

12. The method of claim 11 wherein said measurements and Comparative Data Set are compared in graphical format.

13. The method of claim 1 wherein said comparing step comprises comparing one or more rate constants associated with the data measured in the measuring step with a Comparative Data Set comprising at least one comparative rate constant.

14. The method of claim 1 wherein said comparing step comprises calculating and comparing the rate constants associated with the data measured in the measuring step in the time frame starting from completion of the introducing step up to about 60 seconds after such completion of the introducing step with the measurements taken after about 60 seconds after completion of the introducing step.

15. The method of claim 1 wherein said comparing step comprises fitting the measured data to Equation I in accord with the Equation Constant Fit Method, calculating the constant A associated therewith, and comparing such measured constant A value with a Comparative Data Set comprising at least one comparative constant A value.

16. A method of diagnosing a skin condition comprising evaluating skin in accord with claim 1 and diagnosing an abnormality of the skin based on such evaluation.

17. The method of claim 16 wherein said skin condition is selected from the group consisting of dry skin, psoriasis, eczema, wound of the skin surface layers, diaper rash, dermatitis, burns, and combinations of two or more thereof.

18. A method of treating a skin condition comprising evaluating skin and diagnosing an abnormality thereof in accord with claim 16 and implementing a treatment regimen designed to remedy such abnormality based on such evaluating and diagnosing steps.

19. The method of claim 18 wherein said treatment regimen comprises applying a personal care product to the skin.

20. The method of claim 18 wherein said treatment regimen comprises ingesting an ingestible composition for treatment of the skin.

21. A method of evaluating the efficacy of a treatment regimen comprising evaluating the skin according to the method of claim 1, treating said skin with a treatment regimen for a period of time, and re-evaluating the skin in accord with the method of claim 1.

22. A method of evaluating the efficacy of a treatment regimen comprising introducing a fluid comprising water to the skin; measuring at least one skin barrier property that correlates to the water content of the skin over time, the measuring step comprising taking at least one measurement of the skin barrier property in the time frame starting from completion of the introducing step up to about 60 seconds after such completion of the introducing step and optionally taking a plurality of skin barrier property measurements after about 60 seconds after completion of the introducing step, collecting such measured data into a Pre-treatment Comparative Data Set, treating said skin with a treatment regimen for a period of time, then evaluating the skin according to the method of claim 1 wherein said Comparative Data Set is the Pre-treatment Comparative Data Set.

23. A device for evaluating the skin comprising an energy source, a detector operatively associated with the energy source, a comparing component for determining the relative infant-like barrier properties indicated by the measured data; and a visual or audio display for displaying an indication of relative infant-like barrier properties associated with the measured data.

24. The device of claim 23 further comprising a reservoir from which water may be introduced to the skin.

25. The device of claim 23 further comprising a drying component for removing excess water from the surface of the skin.
